(19) United States
(12) Patent Application Publication
(10) Pub. No.: US 2023/0338718 A1
(43) Pub. Date: Oct. 26, 2023

(54) A SWALLOWABLE CAPSULE DEVICE

(71) Applicant: Novo Nordisk A/S, Bagsvaerd (DK)

(72) Inventors: Jorrit Jeroen Water, Frederiksberg (DK); Brian Mouridsen, Fredensborg (DK); Drago Sticker, Malmoe (SE); Aghiad Ghazal, Koebenhavn SV (DK); Jacob Pyung Hwa Jepsen, Frederiksberg (DK); Peter Herskind, Hilleroed (DK); Cody Edward Cleveland, Koebenhavn K (DK)

(21) Appl. No.: 18/026,643

(22) PCT Filed: Sep. 30, 2021
(86) PCT No.: PCT/EP2021/076893

§ 371 Date: Mar. 16, 2023

(30) Foreign Application Priority Data
Sep. 30, 2020 (EP) 20199482.9

(51) Int. Cl. A61M 31/00; A61M 5/30; A61M 5/19

(52) U.S. Cl. CPC A61M 31/002; A61M 5/30; A61M 5/19

(57) ABSTRACT

The swallowable capsule device (10) comprises a capsule housing (12), a drug outlet (14) arranged relative to the capsule housing (12), a drug reservoir (16) configured to accommodate a drug substance (23) in use, and a needleless jet injector assembly. The needleless jet injector assembly includes a jet injector (22) and an actuator. The jet injector (22) has a plurality of jet nozzles (24) and the actuator is configured to actuate under predetermined conditions to expel the drug substance (23) in use from the drug reservoir out through the drug outlet (14) via the jet nozzles (24) to form a jet stream. Each jet nozzle (24) is configured to expel a separate collimated jet stream (28) having a peak jetting power of 1 W or below, and the plurality of jet nozzles (24) are arranged in a cluster so that the separate collimated jet streams (24) in combination form a contiguous jet stream (20) that is configured to be expelled towards the lumen wall.

This invention relates to a swallowable capsule device suitable for ingestion into a gastrointestinal lumen of a subject.

Devices for oral administration are beneficial for numerous drug delivery applications including delivery of biomacromolecules, such as proteins and other biologics, to the gastrointestinal (GI) tract. Such oral administration is likely to increase patient compliance, reduce drug administration costs and improve the therapeutic outcomes compared with the more invasive forms of drug administration, e.g. subcutaneous injection. Needleless jet based drug delivery is an attractive means of drug delivery for oral administration devices. In comparison to common chemical permeation enhancers resulting in ˜2% bioavailability, a jet-based needle free injection can achieve bioavailability on par with subcutaneous.

Existing jet injector systems for jet drug delivery are known in the art. WO 2020/106,750 A1 include disclosure of ingestible devices that are configured to dispense a dispensable substance in form of a liquid. In some forms the liquid is dispensed as a jet. US 2016/0228646 A1 discloses a particle delivery device, in particular hand-held device, configured to collimate particles entrained in a gas flow stream and to focus the particles such that a beam of particles is perpendicular to a tissue surface.

According to a first aspect of the invention there is provided a swallowable capsule device configured for ingestion into a gastrointestinal lumen of a subject, and for expelling a dose of a drug substance into a lumen wall of the lumen, wherein the capsule device comprises:
a capsule housing,
a drug outlet arranged relative to the capsule housing,
a drug reservoir configured to accommodate a drug substance in use, and
a needleless jet injector assembly including a jet injector and an actuator, the jet injector having a plurality of jet nozzles and the actuator being configured to actuate under predetermined conditions to expel the drug substance in use from the drug reservoir out through the drug outlet via the jet nozzles to form a jet stream,
wherein each jet nozzle is configured to expel a separate collimated jet stream having a peak jetting power of 1 W or below, and
wherein the plurality of jet nozzles are arranged in a cluster so that the separate collimated jet streams in combination form a contiguous jet stream that is configured to be expelled towards the lumen wall.

The inclusion of such a jet injector assembly allows for needle-free jet streams to deliver the drug substance, e.g. an active pharmaceutical ingredient (API), into the lumen wall in use. In this way, the swallowable capsule device does not include sharp needle points, and a mechanism which actuates and retracts the needle is also not required. Moreover, the needle-less jet injection is believed to reduce the pain and/or trauma at the injection site compared to a needle delivery. Existing jet injector assemblies for jet delivery are known in the art. A skilled person would understand how to select an appropriate jet injector assembly that expels the drug substance under predetermined conditions out of the drug outlet and towards the lumen wall. The predetermined conditions may be a time condition, or a trigger based on a specific environment, e.g. the conditions within a certain part of the GI tract. The trigger may also be or include an electronic trigger.

By providing a plurality of jet nozzles whose separate collimated jet streams have a peak jetting power of about 1 W or below (optionally, about 0.8 W or below, optionally still about 0.5 W or below, optionally still about 0.3 W or below) and which combine to form a contiguous jet stream, the risk of tissue perforation is reduced. This is because individual lower peak jetting powers are used which combine to provide a desired total jetting power without that total jetting power being provided from a single powerful jet source, which tends to be harsher on the tissue. Spreading the jetting power between a plurality of jet nozzles also results in a more spread out (slightly dispersed) drug depot in the submucosa space, i.e. the drug depot is spread “horizontally” along the space, rather than the jet stream being completely concentrated at one point from a single jet source, as is the case for a single jet nozzle. This is also true where the jet streams converge to a single target point because the separate jet streams are coming at the injection site from different angles, thus resulting in a more spread out drug depot. Essentially, in the single target point embodiment, the jets stop being jets beyond the point of interaction because they effectively “cancel each other out”, thus resulting in a horizontal spread of substance. Ultimately, this results in a safer drug delivery because the dispersed drug depot reduces the risk of perforation to the tissue on the underside (i.e. opposite to the mucosal on the upperside), which is a risk when using a single powerful jet nozzle.

The term “peak jetting power” is understood in the art of jet injection as meaning the highest jet power that the jet stream experiences during the expelling process. Typically, the power profile for a jet injector reaches a peak jetting power soon after triggering and then experiences a decaying jetting power in the later stage of the expelling process. However, other power profiles are possible, such as one with a peak jetting power at the end of the expelling process.

It will be understood that “spread out” in the context of this application means slightly dispersed. The jet streams are still close enough to one another to considered to be delivering a single drug depot to a single injection site, i.e. via a contiguous jet stream. This differs from a plurality of jet nozzles being used to deliver completely separate drug depots to separate, distinct injection sites.

The use of multiple jet nozzles also helps with the problem of aggregates that can form in the drug substance. Since the drug substance is being forced through multiple jet nozzles, rather than a single larger jet nozzle, this can help to break up aggregates. Moreover, the multiple jet nozzles can act as a filter by blocking larger aggregates from passing through the jet nozzle and being delivered to the tissue. This is particularly true when smaller nozzle orifice diameters are used (as outlined below).

In addition, providing multiple jet nozzles provides for a more reliable drug delivery compared with using a single jet nozzle. This is because if one or more nozzles fail, e.g. they become clogged, there is one or more back up nozzles present to deliver at least some of the drug load.

Preferably, the jet injector assembly is configured to provide the contiguous jet stream having a peak jetting power in a range of about 1 to 5 W.

Optionally, each jet nozzle includes a nozzle orifice through which the drug substance is expelled in use, each nozzle orifice having a diameter of about 10 to 150 μm, optionally about 50 to 150 μm, optionally about 75 to 150 μm, optionally about 10 to 125 μm, optionally about 50 to 125 μm, optionally about 75 to 125 μm.

The centre-to-centre distance between any two nearest neighbouring jet nozzles may be in the range of 0.5 mm and 1.0 mm.

In one embodiment, the jet nozzles are arranged so that the separate collimated jet streams flow substantially parallel to each other from the drug outlet.

In another embodiment, the jet nozzles are arranged so that the separate collimated jet streams converge towards a target point. In such other embodiment, the jet nozzles are angled relative to one another, the angle between each jet nozzle ranging from 30° to 90°.

In each case, the individual collimated jet streams combine to form a contiguous jet stream which is able to deliver a successful drug depot to an injection site.

In some forms, each of the jet nozzles is arranged emerging at an exterior surface portion defined by the capsule housing, e.g. so that each nozzle emerges less than 1 mm from an exterior tissue engaging surface portion of the capsule housing, optionally emerges on the exterior tissue engaging surface portion of the capsule housing.

In certain embodiments of the swallowable capsule device, in use, the drug substance forms a liquid drug substance that is expelled through the drug outlet such that each jet nozzle expels a separate collimated liquid jet stream comprising the drug substance.

In some embodiments, the drug substance is stored in the drug reservoir with the drug substance being provided in the form of a liquid drug substance.

In other embodiments, the drug substance forms a powder in the drug reservoir. In such embodiment, the capsule device may further comprise a second reservoir accommodating a liquid, and wherein the capsule device is configured for mixing the powder and the liquid forming a mixed liquid drug substance that is expellable through the drug outlet. In some embodiments the swallowable capsule device is configured for enabling mixing prior to a patient swallows the capsule device. In other embodiments, the mixing is configured to occur subsequently to swallowing.

In certain embodiments, the drug reservoir of the swallowable capsule device accommodates an expellable volume of the drug substance. The jet injector and the actuator may be configured for expelling substantially all of said expellable volume of the drug substance from the drug reservoir through the drug outlet in the course of a single drug administering action.

In some variants of the capsule device, the actuator also comprises a trigger arrangement for initiating jet injection through the drug outlet. In some forms the trigger arrangement is provided to comprise an environmentally-sensitive mechanism.

In some forms, the capsule device is configured for swallowing by a patient and travelling into a lumen of a GI tract of a patient, such as the stomach, the small intestine or the large intestine, respectively.

The environmentally-sensitive mechanism may in certain embodiments be a GI tract environmentally-sensitive mechanism. The GI tract environmentally-sensitive mechanism may comprise a trigger member, wherein the trigger member is characterised by at least one of the group comprising:
a) the trigger member comprises a material that degrades, erodes and/or dissolves due to a change in pH in the GI tract;
b) the trigger member comprises a material that degrades, erodes and/or dissolves due to a pH in the GI tract;
c) the trigger member comprises a material that degrades, erodes and/or dissolves due to a presence of an enzyme in the GI tract; and
d) the trigger member comprises a material that degrades, erodes and/or dissolves due to a change in concentration of an enzyme in the GI tract.

In further embodiments, the capsule housing may be shaped as an elongated object extending along an axis, the capsule housing defining an interior and having an exterior surface, wherein the interior comprises the drug reservoir, and wherein the drug outlet is disposed at the capsule housing and laterally to the axis to allow needle-less jet injection of the therapeutic substance into the lumen wall.

In still further embodiments, the capsule device may further comprise an expansion assembly comprising an expansion section arranged laterally opposite to and physically separated from the drug outlet, wherein the expansion section is laterally expandable from a non-expanded configuration to an expanded configuration for positioning the delivery outlet against the lumen wall, and wherein the capsule device comprises an expansion control mechanism configured to activate the expansion assembly under a predetermined condition to permit change of the expansion section from the non-expanded configuration to the expanded configuration.

In solution of the prior art, when a dose of the therapeutic substance is expelled by means of a jet nozzle, a pronounced recoil effect is likely to occur which acts to move the jet nozzle away from the targeted tissue area. For example, for capsule devices wherein a single jet nozzle arrangement is located at an end of the housing section, the recoil effect will cause a torque to be exerted onto the capsule device with the result that the jet stream is moved sideways in the course of the expelling action. Hence, for trans-epithelial delivery wherein an initial penetration has been made into a target tissue area by a first portion of the dose, the remaining portion of the dose will be directed towards other tissue areas, potentially leading to penetration of an enlarged tissue area, or potentially leading to loss of therapeutic substance into the lumen of the intestinal tract.

In accordance with the invention, the inclusion of an expansion assembly with an expansion section which is laterally expandable from a non-expanded configuration to an expanded configuration for positioning the delivery outlet against the lumen wall allows for the capsule to be oriented correctly within the intestinal tract ready for jet injection of the therapeutic substance. Further the expansion section serves to fixate the delivery outlet relative to the targeted tissue area during the jet injection process. Thus, the expandable section ensures both proximity of the delivery outlet to the injection site but also ensures that the full dose is delivered through the same penetration opening as initially established at the initiation of the jet delivery action. Moreover, the expansion section assuming a non-expanded configuration allows for ease of swallowing the capsule device for the patient.

Preferred embodiments of the invention will now be described, by way of non-limiting examples, with reference to the accompanying drawings in which:

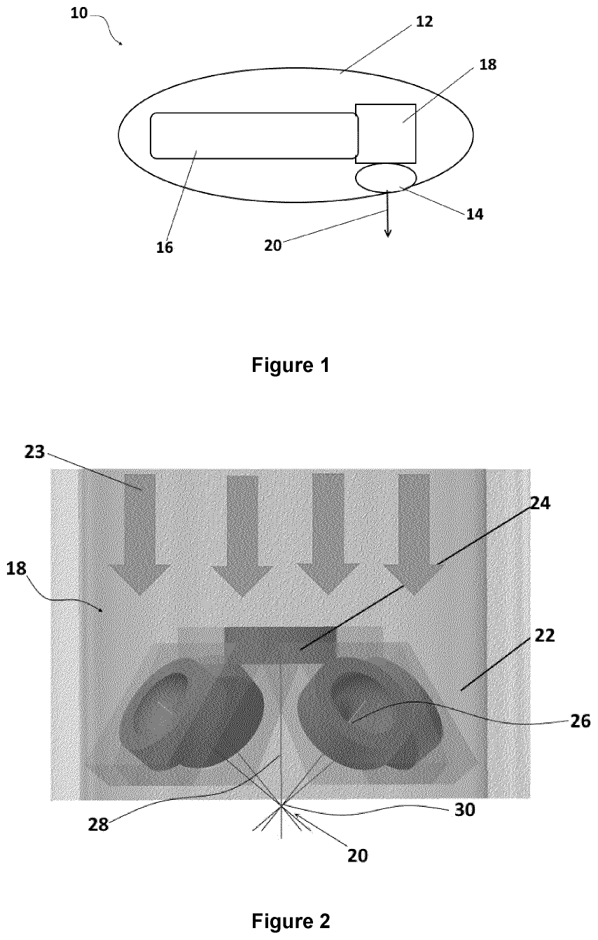

FIG. 1 shows a schematic of a swallowable capsule device according to the invention;

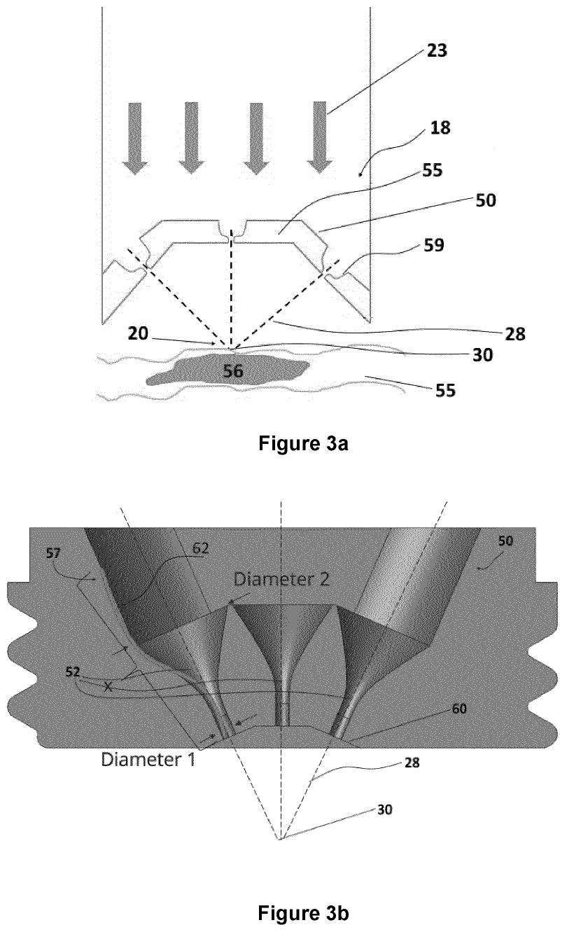

FIG. 2 shows a jet injector according to a first embodiment of the invention;

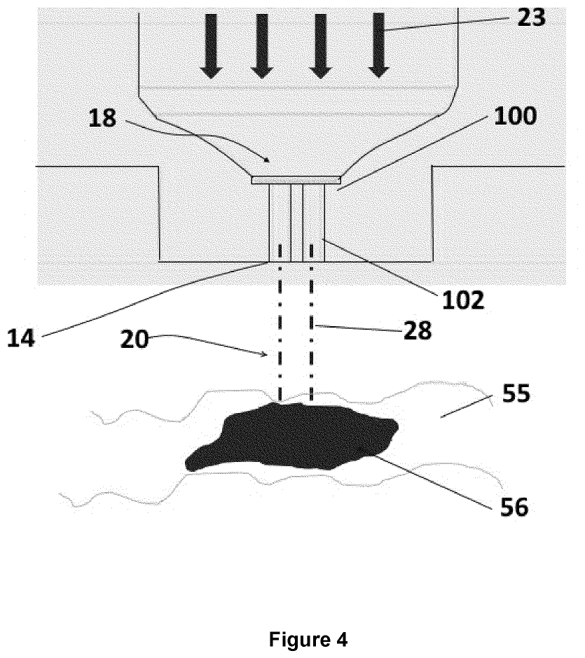

FIG. 3a shows a jet injector according to a second embodiment of the invention;

FIG. 3b shows the jet nozzles of the jet injector of FIG. 3a in more detail;

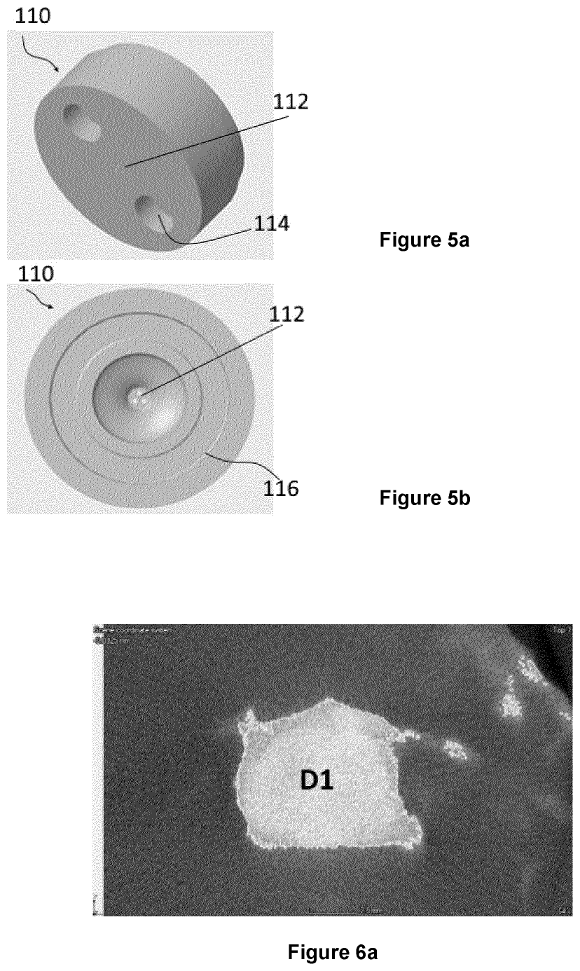

FIG. 4 shows a jet injector according to a third embodiment of the invention;

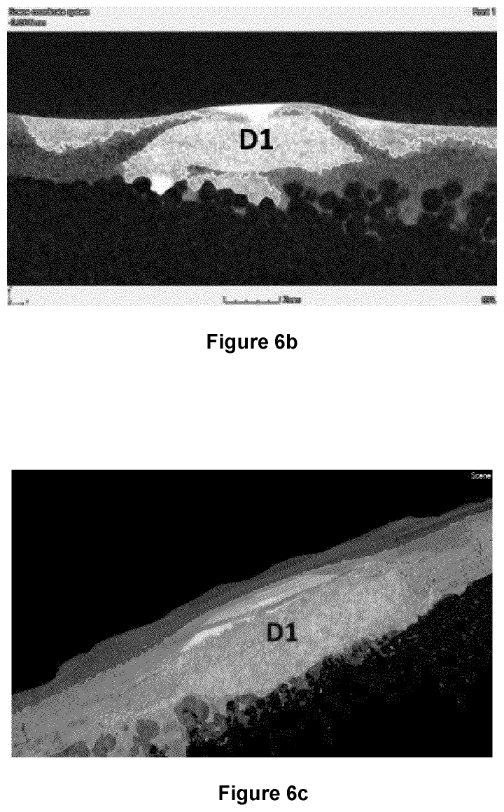

FIGS. 5a and 5b show an example jet nozzle insert for straight jet stream embodiments;

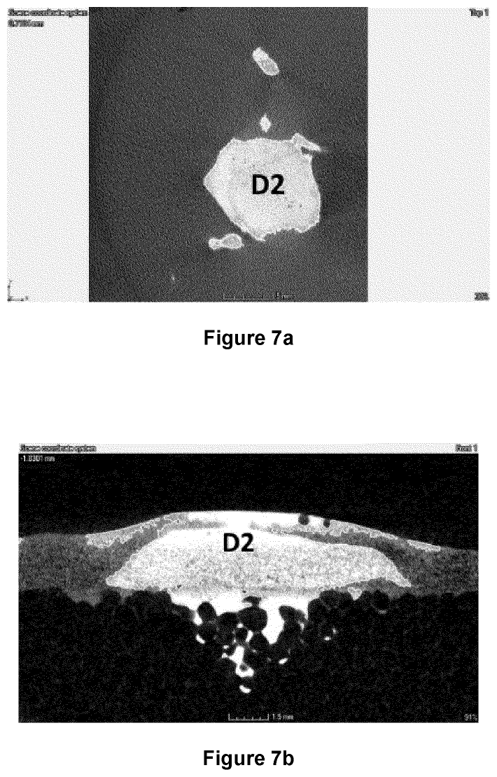

FIGS. 6a to 6c show CT scans of the drug depot resulting from experiment 1, with FIG. 6a showing a top view of the drug depot, FIG. 6b showing a side view of the drug depot and FIG. 6c showing a 3D view of the drug depot;

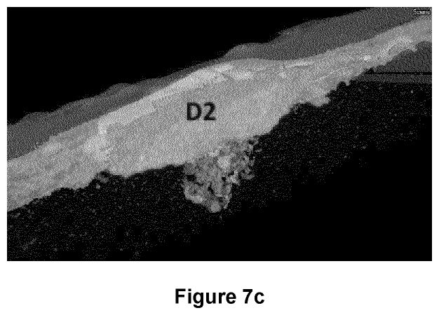

FIGS. 7a to 7c show CT scans of the drug depot resulting from experiment 2, with FIG. 7a showing a top view of the drug depot, FIG. 7b showing a side view of the drug depot and FIG. 7c showing a 3D view of the drug depot,

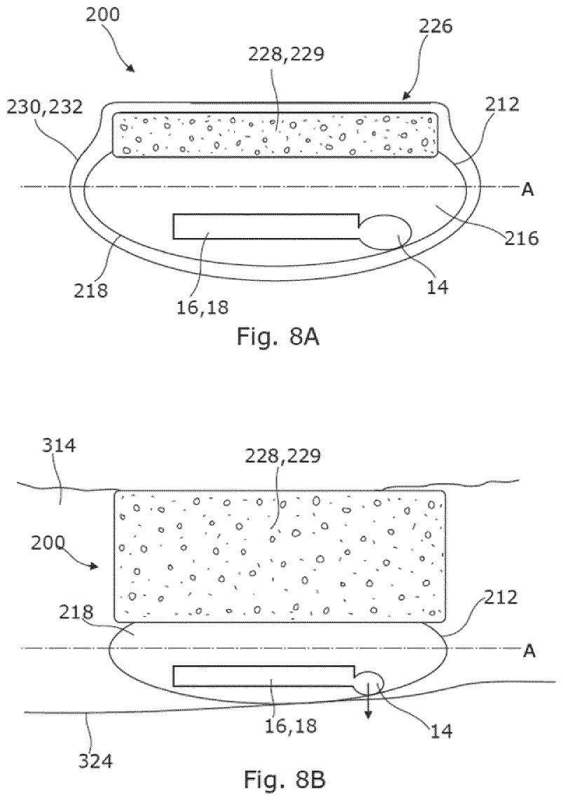

FIG. 8a shows a swallowable capsule device according to a fourth embodiment of the invention with an expansion section in the non-expanded configuration; and

FIG. 8b shows the swallowable capsule device of FIG. 8a with the expansion section in the expanded configuration in vivo.

A swallowable capsule device 10 is shown in FIG. 1. The capsule device is sized and shaped to be suitable for a patient to swallow the capsule device 10 and, particularly, for ingestion into the GI lumen of the patient. The capsule device 10 may be dimensioned as a 00-sized or a 000-sized tablet.

The capsule device 10 includes a capsule housing 12 and a drug outlet 14 that is arranged on the outer surface of the capsule housing 12. In the embodiment shown, there is a single drug outlet 14 but in other embodiments there may be more than one (e.g. one for each jet nozzle).

The capsule device 10 also includes a drug reservoir 16 which is configured to accommodate a drug substance (not shown), and further includes a jet injector assembly 18. As described in more detail below, the jet injector assembly 18 is configured to expel the drug substance via a contiguous jet stream 20 out through the drug outlet 14.

A portion of the jet injector assembly 18 including a jet injector 22 according to a first embodiment of the invention is shown in FIG. 2. The jet injector 22 receives the drug substance 23 from the drug reservoir (not shown). The jet injector 22 includes five jet nozzles 24. Each of the five jet nozzles 24 has a nozzle orifice 26 and is configured to expel a separate collimated jet stream 28 through a respective nozzle orifice 26.

In this embodiment, the jet nozzles 24 are arranged so that the separate collimated jet streams 28 converge towards a target point 30. Specifically, the jet nozzles 24 are angled relative to one another at an angle of 55° so that the collimated jet streams 28 are directed towards the target point 30. As such, the contiguous jet stream 20 in this embodiment is the single target point 30.

In this embodiment, the total input force is 50 N acting on the liquid drug substance 23 and the individual nozzle orifices 26 each have a diameter of 67 μm. This results in a single jetting force (i.e. of each jet nozzle 24) of approximately 0.011 N. This can also be represented in terms of power to be approximately 0.33 W. The five jet nozzles 24 combined power is around 1.67 W at the target point 30.

A portion of the jet injector assembly 18 including a jet injector 50 according to a second embodiment of the invention is shown in FIGS. 3a and 3b. As before, the jet injector 50 receives the drug substance 23 from the drug reservoir (not shown). This time, the jet injector 50 includes three jet nozzles 52, each of which includes a nozzle orifice. Again, each jet nozzle 52 is configured to expel a separate collimated jet stream 28 through a respective nozzle orifice.

The jet nozzles 52 are again angled towards one another so that the separate collimated jet streams 28 converge towards a single target point 30. As shown in FIG. 3a, the target point 30 is in the GI tissue 55 and the contiguous jet stream 20 (which is formed from the converging jet streams 28) forms a drug depot 56 which is spread out along the tissue 55.

FIG. 3a shows a receiving plate 55 which is configured to receive a multi-nozzle insert 57 that includes the three jet nozzles 52. The receiving plate 55 has nozzle receiving portions 59 that are shaped and sized for receiving each jet nozzle 52 of the multi-nozzle insert 57.

The multi-nozzle insert 57 is shown in FIG. 3b and includes the three jet nozzles 52. The multi-nozzle insert 57 is fabricated using CNC machining for accurate milling of each jet nozzle 52 since orientation and point of convergence of said nozzles 52 is critical for this concept to be successful. The nozzle insert 57 shown is made from brass due to its ease of fabrication and rigidness, however any other suitable material may be used.

Each jet nozzle 52 has an outer curved profile 62 which is formed by the outer nozzle wall changing from a larger diameter (i.e. Diameter 2) to a smaller diameter (i.e. Diameter 1, which is akin to the nozzle orifice diameter). The outer curved profile 62 forms a letter “S” shape, as shown in FIG. 3b. This profile 62 shape helps to create a smooth transition of a big volume of liquid to the narrow nozzle with minimal losses. If the change from large (Diameter 2) to small diameter (Diameter 1) is drastic (meaning, the ratio between orifice diameter and nozzle diameter is very large) and the change takes place over a short length (distance x), then the ‘S’ shape will be pronounced causing higher resistances. In extreme cases, this can prevent a jet from forming. However, for a smaller ratio and a longer distance x, then lower resistances and successful jet is ensured. Suitable ratios and distances x which ensure a successful jet for a particular application would be known to the skilled person.

In this embodiment, the jet nozzles 52 are designed and positioned relative to one another so that the target point 30 is located approximately 1 mm away from the outer edge 60 of the nozzle insert 57. In other embodiments, the jet nozzles 52 may be configured such that the target point 30 is closer or further away from the edge 60 of the nozzle insert 57, or indeed flush with the edge 60 of the nozzle insert 57.

In this embodiment, the total input force is 50 N acting on the liquid drug substance 23 and the individual nozzle orifices each have a diameter of 125 μm. The resulting individual jetting force (i.e. of each jet nozzle 52) is approximately 0.038 N. This can also be represented in terms of power to be approximately 1 W. The three jet nozzles 52 combined power is around 3 W at the target point 30.

A portion of the jet injector assembly 18 including a jet injector 100 according to a third embodiment of the invention is shown in FIG. 4. As before, the jet injector 100 receives the drug substance 23 from the drug reservoir (not shown). The jet injector 100 includes two jet nozzles 102. Each of the jet nozzles 102 has a nozzle orifice and is configured to expel a separate collimated jet stream 28 through a respective nozzle orifice.

In this embodiment, the jet nozzles 102 are arranged so that the separate collimated jet streams 28 flow substantially parallel to each other from the drug outlet 14. Specifically, the jet nozzles 102 are arranged side-by-side so that each jet stream 28 exits the drug outlet 14 at essentially 90°. The jet streams 28 may flow parallel to one another but at an angle other than 90° relative to the drug outlet 14.

The contiguous jet stream 20 in this embodiment is the side-by-side jet streams 28. The jet streams 28 are aligned close enough to each other to induce a crack into the target GI tissue 55 to allow the drug to be dosed and situated in the submucosa space.

The centre-to-centre distance between the two jet nozzles 102 is in the range of 0.5 mm to 1 mm.

As shown in FIG. 4, the contiguous jet stream 20 enters the GI tissue 55 and the contiguous jet stream 20 (which is formed from the parallel jet streams 28) forms a drug depot 56 which is spread out along the tissue 55.

FIGS. 5a and 5b show an example jet nozzle insert 110 that can be used for a multi-straight-jet nozzle configuration, much like that shown in FIG. 4.

The jet nozzle insert 110 has three exit orifices 112 at the centre of the insert 110. Each of the exit orifices 112 has a diameter suitable for receiving a jet nozzle. As shown in FIG. 5a, the insert 110 includes two large holes 114 at either side of the exit orifices on the front side of the insert 110. The holes 114 are made so the insert 110 can be tightened using a special device, they have no influence on the jetting process. As shown in FIG. 5b, the insert 110 includes rings 116 for receiving O-rings on the back side of the insert 110, which are used as part of the jetting experimental setup for sealing purposes. Again, the rings have no influence in terms of the jetting process.

It will be understood that the number of jet nozzles may vary to what is described above. There may be more or fewer jet nozzles present. Each jet nozzle may be configured to expel a jet stream at different jet powers. Each jet nozzle may have different nozzle orifice diameters. For the jet nozzles which are arranged angled towards one another, the angles may vary.

For example, the individual jet nozzle power may be 1 W or below, below 1 W, 0.8 W or below, below 0.8 W, 0.5 W or below, below 0.5 W, 0.3 W or below, below 0.3 W. The individual nozzle orifices may have a diameter in the range of 10 to 150 μm, 50 to 150 μm, 10 to 125 μm, 50 to 125 μm, 75 to 150 μm, 75 to 125 μm. The jet nozzles may be angled relative to one another by between 30° to 90°.

In use, a trigger causes an actuator to expel the drug substance from the drug reservoir out through the drug outlet via the jet nozzles. The trigger may be the environment of a specific point in the GI tract, e.g. it might be an enteric coating which dissolves in the intestine to expose the actuator to the GI fluid thus causing an actuation process. The skilled person would understand how to select an appropriate trigger and actuator mechanism and there are several examples existing in this field.

The contiguous jet stream enters the GI tissue at the combined jet force to create a successful drug depot in the tissue.

Existing jet injector systems for jet drug delivery are known in the art. A skilled person would understand how to select an appropriate jet injector that provides the correct peak jetting power to deliver the therapeutic substance into the lumen wall, for example from WO 2020/106,750 A1 (PROGENITY INC).

In particular, the skilled person would understand that during drug delivery into a GI tract of a patient using jet injection, the jet stream created by the jet injector interfaces the lumen of the GI tract and the surface of the GI tract facing the lumen. Ultimately, the drug substance is deposited into the submucosal and/or the mucosal tissue by the substance impacting the mucosal layer of the GI tract (e.g. the epithelial layer and any mucus that may be present on the epithelial layer) as a stable jet stream of fluid with minimal breakup into a spray.

The volume of fluid of the drug substance experiences a peak fluid pressure that generates the jet stream that exits the jet injector with a peak jet velocity. The jet stream impacts the interface of the lumen of the GI tract and the surface of the GI tract facing the lumen with a peak jetting power, peak jet pressure and peak jet force. The skilled person would recognise that these three parameters are interconnected.

The skilled person would understand how to assess and measure the various jet injector characteristics for suitability of use in the described type of jet injection. For example, one way to assess the jet power is to release the jets onto force sensors which measure the force the jet. Based on the force reading, and knowing the area of the nozzle and density of the jetted liquid, the jet velocity can be determined using equation 1. Based on the calculated velocity, the power (in Watts) can be calculated using equation 2. To evaluate the jet pressure (i.e. the pressure at which the jet stream is expelled), equation 3 can be used.

F=ρAV² (eqation1)

P=½ρAV³ (equation 2)

V=\sqrt{2*P_{bar}*100000/ρ*C} (equation 3)
F=Force (N)
ρ=Density (kg/m3)
A=Area of nozzle (m2)
V=Velocity (m/s)
P=power (N)
P_{bar}=Pressure (bar)
C=Nozzle Loss Coefficient (Typically 0.95)

The inventors found that it can be difficult to record the jetting against a force sensor in the multiple jet system as proposed by this invention because the plurality of jet streams tend to collide and, given the geometry of the nozzle, force measurements may be inaccurate.

Therefore, the area of each nozzle can be assessed theoretically. For example, knowing that an impinging force of the jet against the force sensor of 0.15N for a single orifice nozzle of a diameter of 250 μm results in the formation of a good depot. Therefore, it is possible to back calculate the area of each nozzle orifice, thus for a three nozzle system, the diameter of each is roughly 150 μm.

Experiment:

This invention describes the required jetting parameters to ensure that the liquid drug is ejected from the device successfully. It ensures that given a certain pressure, nozzle geometries and arrangement, successful jetting can be achieved. Representative ex vivo experiments were carried out on pig intestine tissue using a triple nozzle system with focussed jet streams.

CT scanning was performed on pig small intestine tissue which was subjected to jetting using the focused nozzles at a total input force of 40N (experiment 1) and 50N (experiment 2).

FIGS. 6a to 6c show the CT scans of the drug depo from experiment 1. FIG. 6a is the top view of the drug depot D1, FIG. 6b is the side view of the drug depot D1 and FIG. 6c is a 3D side view of the drug depot D1.

FIGS. 7a to 7c show CT scans of the drug depot resulting from experiment 2. FIG. 7a shows the top view of the drug depot D2, FIG. 7b shows the side view of the drug depot D2 and FIG. 7c shows a 3D side view of the drug depot D2.

FIGS. 6a to 7c demonstrate the viability of the focused multi-nozzle jetting system. They show efficiency of such a jetting system (volume of drug loaded in the jetting device vs. volume detected in CT varies depending on initial force used).

A capsule device 200 according to a fourth embodiment of the invention is shown in FIGS. 8a and 8b. The capsule device 200 is intended for oral administration and so is sized and shaped accordingly. In particular, the capsule device 200 includes a housing section 212 which is shaped as an elongated object, an oblong shape in this embodiment (although the invention is not restricted to this shape), which extends along an axis A. As shown in FIG. 8b, the elongate axis A is intended to extend along the same elongate axis of the intestinal tract 314 of a patient when the capsule device 200 is in the desired position for therapeutic substance delivery.

The housing section 212 defines an interior 216 that is configured to contain a therapeutic substance (not shown) in use, and has an exterior surface 218.

The capsule device 200 also includes a delivery outlet 14 comprising a plurality of jet nozzles that is positioned laterally to the axis A. The capsule device 200 further includes a delivery assembly 16, 18 that includes a jet injector (not shown) which is configured to deliver, in use, the therapeutic substance through the delivery outlet 14 and into the lumen wall 324 by jet injection.

The capsule device 200 further includes an expansion assembly 26 that includes an expansion section 228. The expansion section 228 is arranged laterally opposite to and physically separate from the delivery outlet 14. The expansion section 228 is laterally expandable from a non-expanded configuration (as shown in FIG. 8a) to an expanded configuration (as shown in FIG. 8b). As illustrated in FIG. 8b, the expansion section 228 being in the expanded configuration orients the capsule device 200 within the tract 314 so that the delivery outlet 14 is positioned against the lumen wall 324.

Preferably, the expanded section 228 extends along the majority, or full, length of the housing section 212.

In this embodiment, the expansion section 228 is in the form of a sponge 229, which assumes the expanded configuration when wetted.

The capsule device 200 also includes an expansion control mechanism 230 which, in this embodiment, is in the form of an enteric coating 232. The coating 232 surrounds the whole capsule device 200, although this may not be the case in other embodiments, e.g. it may only cover the expansion section 228. The enteric coating 232 is configured to dissolve when it is exposed to the intestinal fluid in the tract 314 such that it exposes the expansion section 228, i.e. the sponge 229, to the intestinal fluid, thus activating expansion of the sponge 229.

The enteric coating 232 (sometimes referred to as a gastro-resistant coating) is a barrier that resists breakdown before it reaches the small intestine, and then dissolves due to the characteristics (e.g. pH, pressure, acidity, temperature, etc.) of the small intestine. The coating may take another form to dissolve in another part of the GI tract 314, depending on where substance delivery is required.

The expansion control mechanism 30 may be another form of dissolvable trigger such as a time-dependent coating which dissolves after a predetermined period of time. The expansion control mechanism 230 may include a sensor which detects a desired position within the GI tract 314 to then activate the expansion section 228.

Moreover, the expansion control mechanism 30 may include a combination of triggers to activate the expansion section 228.

In this embodiment, the sponge 229 is shown as a rectangular solid shape when expanded (although it may be a different solid shape such as an oval, circle or square). The sponge 229 in this embodiment is biodegradable such that is degrades over time, e.g. in nature.

The length of the expansion section 28 should be such the capsule device 200 will start to turn and align with the longitudinal axis of the lumen 314 when the expansion section 228 is triggered. Moreover, the overall width of the capsule device 200 when the expansion section 228 is in the expanded configuration should be such that it presses the delivery outlet 14 against the lumen wall 324, thus allowing injection to take place at the intended injection site. The expansion section 228, however, should not expand so much that it would cause great discomfort to the patient or damage the lumen wall in any way. The length of the expanded section 212 preferably should be equal to or larger than the diameter of the lumen 314. For example, it could be 2×maximum diameter of the lumen. However, there may be instances where the length of the expanded section 312 is less than the lumen diameter. The overall width of the device 10 with the expansion section 228 in the expanded configuration is preferably equal to or more than the maximum lumen diameter.

The capsule device 10 in this example has the following dimensions. The tablet housing 212 has a length of about 25 mm and a width of about 8.5 mm. The width of the expansion section in the expanded configuration is around 30 mm and the length in the expanded configuration is between 30-70 mm, preferably between 50-70 mm. The capsule device may be configured as a 00-sized capsule or a 000-sized capsule.

It will be understood that these dimensions are for illustrational purposes only and any suitable dimensions may be chosen depending on the intended injection site and/or the patient in question. For example, the dimensions described above are intended for use in an adult small intestine, which typically has a dimeter of between 25 to 30 mm. However, a child's small intestine diameter is smaller and so different dimensions can be chosen (particularly for the width and length of the expanded section) depending on the patient's age bracket. Data regarding the dimensions of an intended injection site, e.g. the intestine, would be readily available to a skilled person.

The description in relation to the dimensions of the capsule device 200 and expanded section 228 applies to the other embodiments of the invention described below.

In use, the capsule device 200 is swallowed by a patient and moves along a lumen of the intestinal tract 314 of the patient. When the capsule device 200 reaches the small intestine tract, the enteric coating 32 begins to dissolve due to the conditions of the small intestine. Such dissolving results in the expansion section 228, i.e. the sponge 229, being exposed to the small intestine fluid.

The fluid wets the sponge 229 which causes the sponge 229 to expand from its non-expanded configuration to its expanded configuration. The size and positioning of the expanded sponge 229 relative to the size of the lumen causes the capsule device 10 to position itself longitudinally along the longitudinal axis of the lumen. In doing so, the delivery outlet 14 is positioned against the lumen wall 324, ready for jet injection by the delivery assembly 222.

Jet injection is then carried out by the jet injector to deliver the therapeutic substance into the lumen wall 324 of the patient.

The length of the capsule device 200 is just larger than the lumen diameter and/or the lumen diameter when in a contracted configuration (e.g. when no food is passing through) so that the capsule device 200 cannot orient itself vertically within the lumen (i.e. the capsule device longitudinal axis A cannot lie perpendicular to the longitudinal axis of the lumen). Therefore, a portion of the sponge 229 (which preferably extends along the majority or full length of the housing section 212, or extends beyond the length of the housing section 212), when expanded, will push against the lumen wall 324 so as to orient the capsule device 200 as desired (i.e. as shown in FIG. 8b).

1. A swallowable capsule device configured for ingestion into a gastrointestinal lumen of a subject, and for expelling a dose of a drug substance into a lumen wall of the lumen, wherein the capsule device comprises:
a capsule housing,
a drug outlet arranged relative to the capsule housing,
a drug reservoir configured to accommodate a drug substance in use, and
a needleless jet injector assembly including a jet injector and an actuator, the jet injector having a plurality of jet nozzles and the actuator being configured to actuate under predetermined conditions to expel the drug substance in use from the drug reservoir out through the drug outlet via the jet nozzles to form a jet stream,
wherein each jet nozzle is configured to expel a separate collimated jet stream having a peak jetting power of 1 W or below, and
wherein the plurality of jet nozzles are arranged in a cluster so that the separate collimated jet streams in combination form a contiguous jet stream that is configured to be expelled towards the lumen wall.

2. The swallowable capsule device as in claim 1, wherein in use, the drug substance forms a liquid drug substance that is expelled through the drug outlet such that each jet nozzle expels a separate collimated liquid jet stream comprising the drug substance.

3. The swallowable capsule device as in claim 2, wherein the drug substance forms a liquid drug substance accommodated in the drug reservoir.

4. The swallowable capsule device as in claim 2, wherein the drug substance forms a powder in the drug reservoir, wherein the capsule device further comprises a second reservoir accommodating a liquid, and wherein the capsule device is configured for mixing the powder and the liquid forming a mixed liquid drug substance that is expellable through the drug outlet.

5. The swallowable capsule device as in claim 1, wherein the drug reservoir accommodates an expellable volume of the drug substance, and wherein the jet injector and the actuator are configured for expelling substantially all of said expellable volume of the drug substance from the drug reservoir through the drug outlet as a single drug administering action.

6. The swallowable capsule device as in claim 1, wherein each jet nozzle is configured to provide a separate collimated jet stream having a peak jetting power of 0.8 W or below.

7. The swallowable capsule device as in claim 1, wherein the jet injector assembly is configured to provide the contiguous jet stream having a peak jetting power in a range of 1 to 5 W.

8. The swallowable capsule device as in claim 1, wherein each jet nozzle includes a nozzle orifice through which the drug substance is expelled in use, each nozzle orifice having a diameter of 10 to 150 μm.

9. The swallowable capsule device as in claim 1, wherein each jet nozzle includes a nozzle orifice through which the drug substance is expelled in use, each nozzle orifice having a diameter of 50 to 125 μm.

10. The swallowable capsule device as in claim 1, wherein the centre-to-centre distance between any two nearest neighbouring jet nozzles is in the range of 0.5 mm and 1.0 mm.

11. The swallowable capsule device as in claim 1, wherein the jet nozzles are arranged so that the separate collimated jet streams flow substantially parallel to each other from the drug outlet.

12. The swallowable capsule device as in claim 1, wherein the jet nozzles are arranged so that the separate collimated jet streams converge towards a target point.

13. The swallowable capsule device as in claim 12, wherein the jet nozzles are angled relative to one another, the angle between each jet nozzle ranging from 30° to 90°.
